(19) United States
(12) Patent Application Publication
(10) Pub. No.: US 2016/0377637 A1
(43) Pub. Date: Dec. 29, 2016

(54) METHOD AND APPARATUS FOR DIAGNOSING BONE TISSUE CONDITIONS

(71) Applicant: THE REGENTS OF THE UNIVERSITY OF MICHIGAN, Ann Arbor, MI (US)

(72) Inventors: Francis W.l. Esmonde-White, Ann Arbor, MI (US); Karen A. Esmonde-White, Ann Arbor, MI (US); Michael D. Morris, Ann Arbor, MI (US); Blake J. Roessler, Ann Arbor, MI (US)

(73) Assignee: THE REGENTS OF THE UNIVERSITY OF MICHIGAN, Ann Arbor, MI (US)

(21) Appl. No.: 15/116,533

(22) PCT Filed: Feb. 5, 2015
(86) PCT No.: PCT/US15/14571

§ 371 Date: Aug. 4, 2016

Related U.S. Application Data
(60) Provisional application No. 61/936,261, filed on Feb. 5, 2014.

(51) Int. Cl. G01N 33/84; G01N 21/65; C12Q 1/04

(52) U.S. Cl. CPC G01N 33/84; C12Q 1/04; G01N 21/65; G01N 2800/10; G01N 2201/12; G01N 2201/08

(57) ABSTRACT

Example methods and apparatus are disclosed for diagnosing or assisting a diagnosis of a bone tissue condition. A specimen associated with bone tissue suspected of being infected is irradiated using a monochromatic light source. The specimen may be irradiated in vivo or ex vivo, and/or within a growth medium. Light scattered during the irradiation is gathered and its Raman spectral content is determined to detect one or more pathological calcium phosphate minerals, such as brushite and uncarbonated apatite, resulting from a conversion of carbonated-apatite in the presence of bacteria.

### CROSS-REFERENCE TO RELATED APPLICATIONS

This application claims the benefit of priority to U.S. Provisional Patent Application No. 61/936,261, entitled “Method and Apparatus for Diagnosing Bone Tissue Conditions,” and filed on Feb. 5, 2014, the entire disclosure of which is hereby incorporated by reference herein in its entirety and for all purposes.

### STATEMENT REGARDING FEDERALLY SPONSORED RESEARCH OR DEVELOPMENT

This invention was made with Government support under grant numbers T32-AR-007080, R01-AR-055222, R01-AR-047969, and R21-EB-101026, awarded by the National Institutes of Health. The Government may own certain rights in this invention.

### FIELD OF THE DISCLOSURE

The present disclosure generally relates to medical diagnostic apparatus and methods, and more particularly to apparatus and methods that may be used to help diagnose the condition of bone tissue.

### BACKGROUND

Osteomyelitis is an infection to the bone caused by bacteria or other microorganisms. Bacteria may enter the bone through an injury such as an open fracture, through penetration of a contaminated object, or during orthopedic surgery. In addition, bacteria and microorganisms from an infected part of the body may be carried through the bloodstream to the bone. Individuals that are susceptible to injury or have an illness affecting the body's immune system are generally at a higher risk of developing a bone infection.

A bone biopsy is a reliable method used for detecting and diagnosing osteomyelitis. In some suspected cases of bone infection, a sample of bone tissue is removed for examination and analysis. Often times the bone sample may be taken by medical personnel during surgery while the individual is under local or general anesthetic. Needle aspiration or needle biopsy is another approach in which samples of bone tissue are obtained by using one or more hollow needles. Following either of these procedures, the bone tissue samples are sent to a laboratory for analysis to determine their condition.

A bone infection may also be detected through blood tests, e.g., a white blood cell count and a red blood cell sedimentation rate, which are administered by drawing blood through a needle inserted into a vein. The blood samples are then sent to a laboratory for analysis and the results are typically available within a few days. A blood culture or sample, joint fluid, or pus can also be sent to a laboratory for analysis to assist in the identification of causative organisms.

Various imaging devices can also be used to diagnose osteomyelitis. An x-ray is often the first diagnostic technique employed when a bone infection is suspected. However, because an x-ray may not show changes in the bone until several weeks after an infection has begun, other imaging devices are often used as well. Magnetic Resonance Imaging (MRI) is a relatively expensive technique capable of distinguishing osteomyelitis from bone tumors or dead tissue, but this procedure may not be appropriate for use in all cases. A computed tomography scan (CT) can also be performed, although the results are sometimes less specific than those obtained with MRI. A radionuclide bone scan can also be administered and is especially useful for revealing metabolic changes in the bone caused by fractures or disease well before they may be detected with a conventional x-ray. The radionuclide bone scan may produce positive results in 24 to 48 hours after symptoms begin and is performed by giving the patient an intravenous injection of a radioactive material named technetium. Several hours after the technetium has been introduced into the body, it becomes concentrated in the bone tissue and scanning images are then taken.

While each of these techniques for detecting bone infections may be more suitable for one particular application or another, most are invasive to some degree and the procedures can be very uncomfortable for patients. Many of these detection techniques require advanced scheduling and may also require hours or days to obtain the results. In cases of sepsis and hospital-acquired infections, faster detection methods would improve treatment decisions in these situations, and ultimately, patient outcomes.

### SUMMARY OF THE DISCLOSURE

Described herein are example methods and apparatus for diagnosing or facilitating a diagnosis of a bone tissue condition. In one example method, a specimen associated with bone tissue suspected of being infected is irradiated using a light source. The specimen may be irradiated in vivo or ex vivo. Alternatively, the specimen may include a sample material associated with the suspect bone tissue and placed within a growth medium, which is then irradiated. In either instance, the light source, which may be substantially monochromatic, is deflected and scattered during the irradiation and the resulting signal is collected. The spectral content of the collected scattered light is determined and used, at least in part, to determine the condition of the bone.

In another example embodiment, a method for determining whether a bone tissue is infected may include preparing a specimen associated with bone tissue suspected of infection, monitoring a specimen for bacteria, and indicating a presence of bacteria. If desired, the specimen may include a sample material obtained from an area proximate the bone tissue suspected of being infected, placing the sample material within a growth medium, and irradiating the sample material and the growth medium with a light source.

A further example embodiment is directed to an apparatus for use with a method of determining whether a bone tissue is infected wherein a sample material associated with the bone tissue is placed within an apatite-impregnated aqueous growth medium and irradiated to detect the presence of brushite, which may result from a conversion of carbonated-apatite in the presence of bacteria. The apparatus includes a substantially monochromatic light source to irradiate the sample material within the apatite-impregnated aqueous growth medium, a light receiver to receive light scattered from the irradiated sample material and/or growth medium, and a Raman spectrum analyzer optically coupled to receive scattered light received by the light receiver. The Raman spectrum analyzer is configured to generate Raman spectral content information associated with the received scattered light. A computing device communicatively coupled to the Raman spectrum analyzer is configured to generate diagnostic information indicative of whether brushite is present within the sample material and/or growth medium. A display device may be operatively coupled with the apparatus to indicate the condition of the bone tissue.

### BRIEF DESCRIPTION OF THE DRAWINGS

FIG. 1 is a block diagram of an example apparatus used to determine a bone tissue condition, according to an embodiment.

FIG. 2 is a flow diagram of an example method for determining a bone tissue condition, according to an embodiment.

FIG. 3 is a block diagram of a computer that may be used with the example apparatus of FIG. 1, according to an embodiment.

FIG. 4 is a table of results of a clinical study.

### DETAILED DESCRIPTION

Calcium phosphate minerals are present throughout the body in both health and disease and the most common calcium phosphate mineral found within the body is carbonated apatite, which is found in bone tissue. Through the normal process of bone resorption and formation, carbonated apatite is produced through a series of phase transformations from calcium phosphate precursors. These precursors may include calcium pyrophosphate, amorphous calcium phosphate, octacalcium phosphate, and dicalcium phosphate dihydrate (DCPD, also called brushite). Under normal in vivo conditions, these precursors rapidly convert to carbonated apatite and are considered transient, unstable mineral species.

A bone infection, which may have been caused by an infection of adjacent soft tissue at a wound site, trauma, or a blood borne infection, creates a localized acidic environment. It is believed that, in this acidic environment, carbonated apatite dissolves and converts to pathological calcium phosphate minerals, such as brushite and uncarbonated apatite, upon re-precipitation. Pathological calcium phosphate minerals, such as brushite and uncarbonated apatite, formed under these conditions are stable and can be detected using Raman spectroscopy. Brushite has a Raman spectrum distinct from that of carbonated apatite, and this characteristic (e.g., characteristic Raman bands) may be used to identify trace amounts of brushite in bone tissue or sample materials associated with bone tissue. Similarly, uncarbonated apatite has a Raman spectrum distinct from that of carbonated apatite, and this characteristic (e.g., characteristic Raman bands) may be used to identify trace amounts of uncarbonated apatite in bone tissue or sample materials associated with bone tissue. Other calcium phosphate minerals not found under normal (i.e., healthy) physiological conditions including, by way of example and not limitation, phosphate minerals found at pH values more acidic than physiological pH and carbonated apatite outside the carbonation range of normal bone, may also be detected using Raman spectroscopy.

A clinical study of bone fragments infected with diabetic osteomyelitis revealed the identification of Pathological calcium phosphate minerals, such as brushite and uncarbonated apatite, using Raman spectroscopy. Raman spectroscopy is particularly well-suited for combining with other analytical techniques given that it provides a non-destructive analysis requiring little or no sample preparation and is capable of studying aqueous and solid samples. It is believed that the presence of pathological calcium phosphate minerals, such as brushite and uncarbonated apatite, in bone is evidence of a local acidic pH ˜4.5, which is indicative of bacterial infection. Pathological calcium phosphate minerals, such as brushite and uncarbonated apatite, act similarly to secondary biomarkers used in clinical studies and their Raman signatures may be used to achieve a detection limit that is lower than currently used detection limits and techniques.

Detecting the presence of bacteria is typically determined by measuring pH changes of an entire culture medium, which requires the bacteria to grow into colonies large enough to affect the entire medium volume. In contrast, the conversion of carbonated apatite to pathological calcium phosphate minerals, such as brushite and uncarbonated apatite, may be monitored as a sensitive measurement of bacterial content. In some embodiments, carbonated apatite may be incorporated into an aqueous bacterial growth medium to measure local pH changes associated with small numbers of bacteria. In some embodiments, carbonated apatite is present in a growth medium, and bacteria present anywhere within the medium that converted even a minimal amount of apatite to pathological calcium phosphate minerals, such as brushite and uncarbonated apatite, may provide a quickly distinguishable indication of bacterial infection.

In some embodiments, an instrument capable of monitoring formation of pathological calcium phosphate minerals, e.g., brushite and uncarbonated apatite, such as a wide-beam Raman measurement, may be used to scan over a bacterial culture bottle and/or a specimen associated with bone tissue and provide real-time or close to real-time feedback. In some embodiments, an apatite-impregnated growth medium for disposable bottles may be less expensive as compared to sophisticated molecular probes, such as fluorescent-tagged beads, which might otherwise be used to detect bacterial surface proteins or other unique markers of disease. In addition, different media (including different antibiotic agents) may also be used to differentiate between bacteria by their ability to grow in the media, particularly gram-positive vs. gram-negative.

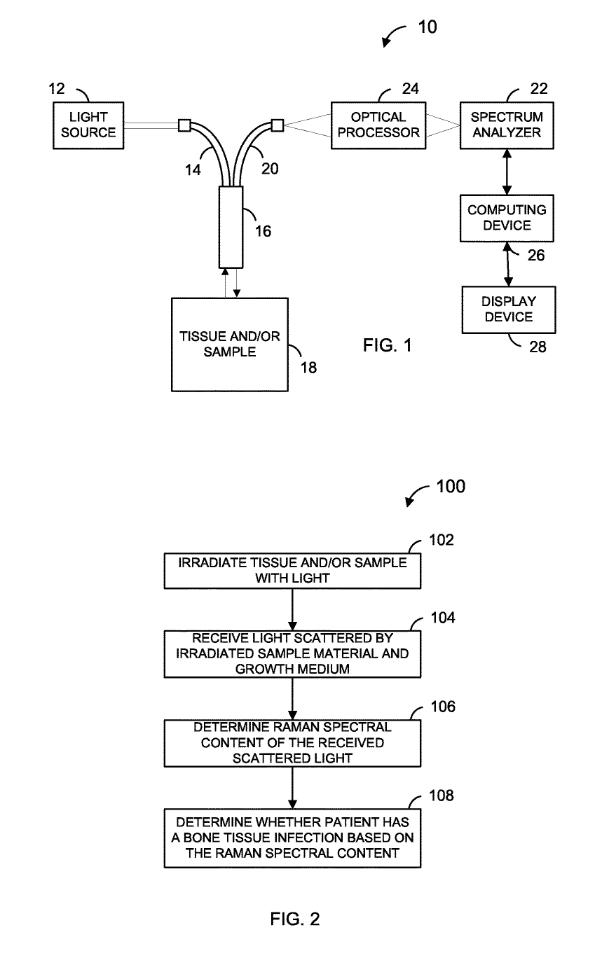

Turning now to FIG. 1, a block diagram of an example apparatus 10 that may be used to assist in the diagnosis of a bone tissue condition, such as an infection, e.g., osteomyelitis, or other related disorder is depicted. The example apparatus 10, which may be used for a Raman spectrometry analysis of a specimen 18 associated with a suspect bone tissue, includes a light source 12 optically coupled to one or more optical fibers 14. For Raman spectrometry, the light source 12 may comprise a laser, for example, that generates substantially monochromatic light. The optical fiber(s) 14 is operatively coupled to an optical probe 16, which may be position proximate to the specimen 18.

The specimen 18 may be a sample material associated with a portion of bone tissue suspected of being infected. In particular, the sample material may be a portion of in vivo bone tissue, which may be irradiated non-invasively through the skin with the light generated by the light source 12, or which may be exposed by an incision and irradiated directly by the light source 12. Alternatively, the sample material may be a portion of ex vivo bone tissue removed as a biopsy and irradiated directly or within a growth medium by the light source 12. Additionally, the specimen 18 may be a swabbed sample material of an area proximate to an associated portion of suspect bone tissue, which may then be placed within a growth medium wherein the optical probe 16 may be used to irradiate the sample material and/or the growth medium with the light generated by the light source 12.

In embodiments that utilize a growth medium, the growth medium may be an aqueous substance impregnated with carbonated apatite. The carbonated apatite impregnated within the aqueous growth medium will convert to pathological calcium phosphate minerals, such as brushite and uncarbonated apatite, in the presence of bacteria. Therefore, if bacteria is present on the sample material (e.g., in vivo sample or ex vivo sample) associated with the portion of bone tissue suspected of being infected, pathological calcium phosphate minerals, such as brushite and uncarbonated apatite, will form and be present as a result of its conversion from carbonated apatite within the growth medium. The Raman spectral content of the collected scattered light is determined and used, at least in part, to determine the presence of one or more pathological calcium phosphate minerals, such as brushite and uncarbonated apatite.

The optical probe 16 may also be operatively coupled to one more other optical fibers 20. The optical probe 16 may be used to collect light scattered by the specimen 18 and to transmit the collected scattered light through the optical fiber(s) 20. The optical fiber(s) 20 may be operatively coupled to a Raman spectrum analyzer 22 via an optical processor 24, which may include one or more lenses and/or one or more filters. The Raman spectrum analyzer 22 may include, for example, a Raman spectrograph operatively coupled to an array of optical detectors, and is communicatively coupled to a computing device 26. A display device 28 may be incorporated with the computing device 26 to display the Raman spectrograph and/or indicate the presence of one or more pathological calcium phosphate minerals, such as brushite and uncarbonated apatite, and/or a bone infection.

Any suitable Raman probe may be utilized as the probe 16. In one embodiment, a probe such as described in U.S. Pat. No. 8,054,463 is utilized, and at least some techniques and/or apparatus described in U.S. Pat. No. 8,054,463 are utilized. U.S. Pat. No. 8,054,463 is expressly incorporated by reference herein.

FIG. 2 is a flow diagram of an example method for determining a condition related to the bone tissue of a patient, according to an embodiment. The method 100, which may be implemented by a suitable apparatus such as the example apparatus 10 of FIG. 1 or another suitable apparatus, includes irradiating a specimen 18 associated with a bone tissue suspected of being infected such as described above. FIG. 2 is discussed with reference to FIG. 1 merely for explanatory purposes.

The optical probe 16 may be used to irradiate the specimen 18 with substantially monochromatic light generated by the light source 12 at a block 102. Light scattered by the specimen 18 during irradiation may be collected by the optical probe 16 at a block 104. Raman spectral content information associated with the collected scattered light is generated at a block 106. The scattered light collected by the optical probe 16 or the optical fiber 20 may be provided to the Raman spectrum analyzer 22 via the optical processor 24. The Raman spectrum analyzer 22 may then generate Raman spectral content information associated with the light received by the Raman spectrum analyzer 22.

In Raman spectrometry, the collected scattered light may include light at wavelengths shifted from the wavelength of the incident light. The Raman spectrum of the collected light scattered from the sample material and/or growth medium 18 may be indicative of the physico-chemical state of the bone tissue and/or the presence of bacteria. The Raman spectrum of the sample material and/or growth medium 18 may include bands indicative of various components of the tissue and/or sample including phosphate of bone mineral, carbonate of bone mineral, interstial water, residual water, hydroxide of the bone mineral, pathological calcium phosphate minerals, such as brushite and uncarbonated apatite, etc. The wavelength at which a band is located may be indicative of the component of the bone mineral or matrix to which it corresponds. The height and/or intensity of a band may be indicative of the amount of the corresponding component of the sample material. In some embodiments, area(s) under one or more bands, height(s) of one or more bands, ratio(s) of multiple band areas, ratio(s) of multiple band heights, etc., in the Raman spectrum of the sample material may be used to determine whether one or more pathological calcium phosphate minerals, such as brushite and uncarbonated apatite, are present, and/or whether bacteria is present.

At a block 108, it is determined whether the patient has a bone tissue disorder based on the Raman spectral content information generated at block 106. For example, the computing device 26 may receive Raman spectral content information from the Raman spectrum analyzer 22. The computing device 26 may then generate an indicator of whether the patient has a bone tissue disorder. That is, the computing device 26 may generate an indication based on the Raman spectral content information generated at block 106. The indication may be an audible or visible alarm, a printout on a display screen or paper, a message, etc., that may be used to indicate the presence of one or more pathological calcium phosphate minerals, such as brushite and uncarbonated apatite, which indicate a bone tissue condition or disorder, such as osteomyelitis, a bacterial infection, etc.

In some embodiments, the determination of block 108 comprises determining whether the bone tissue of the patient is a bacterially infected. The manner in which a bone tissue infection is determined may vary according to the environment in which the determination is made. Similarly, different embodiments of the apparatus 10 for determining a bone tissue disorder may vary in design according to the environment in which the apparatus is to be used. For example, an apparatus to be used in a clinical setting may be designed to obtain spectrum information more quickly as compared to an apparatus to be used in a laboratory setting. Also, it is to be understood by one of ordinary skill in the art that the specificity and sensitivity as related to the detection limits associated with the determination of bacteria present in relation to the amount of one or more pathological calcium phosphate minerals, such as brushite and uncarbonated apatite, existing within the examined tissue, sample material, aqueous carbonated apatite growth medium, etc. may be adjustable by the operator of the apparatus 10.

Referring again to FIG. 1, many suitable types of light sources 12 and wavelengths may be employed. A variety of substantially monochromatic light sources can be used, including commercially available light sources. In general, a wavelength of a light source may be chosen based on various factors including one or more of a desired depth of penetration through skin or growth medium (if desired), availability of photo detectors capable of detecting light at and near the wavelength, efficiency of photo detectors, cost, manufacturability, lifetime, stability, scattering efficiency, penetration depth, etc.

With regard to Raman spectrometry, a substantially monochromatic light source may be used. A light source having a particular wavelength may be suitable for cases requiring penetration into skin tissue or the growth medium. As discussed above, in some embodiments, at least some techniques and/or apparatus described in U.S. Pat. No. 8,054,463, or similar techniques and/or apparatus, are utilized. In some embodiments, if the bone tissue is to be exposed by incision, or if biopsied bone tissue is to be examined, other suitable wavelengths, techniques, apparatus, etc., may be employed. In general, near-infrared wavelengths provide better depth of penetration into tissue, sample, growth medium, etc. On the other hand, as wavelengths lengthen, they may begin to fall outside the response range of silicon photo detectors, which have much better signal-to-noise ratios than other currently available detectors.

With regard to the optical probe 16, any of variety optical probes designed for Raman spectroscopy may be used, including commercially available optical probes. Some commercially available fiber optic probes include filters to reject Raman scatter generated within the excitation fiber and/or to attenuate laser light entering the collection fiber or fibers. Loosely focused light may help eliminate or minimize patient discomfort as compared to tightly focused light. As is known to those of ordinary skill in the art, loosely focused light may be achieved by a variety of techniques including multimode delivery fibers and a long focal length excitation/collection lens(es). As discussed above, in some embodiments, at least some techniques and/or apparatus described in U.S. Pat. No. 8,054,463, or similar techniques and/or apparatus, are utilized.

Existing commercially available fiber optic probes may be modified, or new probes developed, to maximize collection efficiency of light originating at depths of 1 millimeter or more below the surface of a highly scattering medium, such as tissue or a growth medium. Such modified, or newly developed probes, may offer better signal-to-noise ratios and/or faster data collection. The probe may be modified or may be coupled to another device to help maintain a consistent distance between the probe and the tissue or growth medium, which may help to keep the system in focus and help maximize the collected signal.

If the specimen of bone tissue is to be irradiated via an incision (and/or the scattered light is to be collected via an incision), relay optics may be coupled to, or incorporated in, a needle. In general, the size and the number of fibers used should be appropriate to fit into the needle. The diameter of the excitation/collection lens or lenses used in such an embodiment could be small to help minimize the size of the incision. Lenses having larger or smaller diameters could be used as well. The lens(es) and or optical fibers could be incorporated into a hypodermic needle.

Alternatively, it may be possible to use a microprobe or microscope (e.g., a modified epi-fluorescence microscope) instead of the optical probe 16 of FIG. 1. In this case, the optical fiber 14 and/or the optical fiber 20 may be omitted.

The optical processor 24 may include one or more lenses for focusing the collected scattered light. The optical processor 24 may also include one or more filters to attenuate laser light. Although shown separate from the spectrum analyzer 22, some or all of the optical processor 24 may optionally be a component of the spectrum analyzer 22.

The computing device 26 may comprise, for example, an analog circuit, a digital circuit, a mixed analog and digital circuit, a processor with associated memory, a desktop computer, a laptop computer, a tablet PC, a personal digital assistant, a workstation, a server, a mainframe, etc. The computing device 26 may be communicatively coupled to the spectrum analyzer 22 via a wired connection (e.g., wires, a cable, a wired local area network (LAN), etc.) or a wireless connection (a BLUETOOTH™ link, a wireless LAN, an IR link, etc.). In some embodiments, the Raman spectral content information generated by the Raman spectrum analyzer 22 may be stored on a portable memory device, (e.g., memory disk, memory stick, a compact disk (CD), digital video disk (DVD)), and then transferred to the computing device 26 via the disk. Although the Raman spectrum analyzer 22 and the computer 26 are illustrated in FIG. 1 as separate devices, in some embodiments the Raman spectrum analyzer 22 and the computing device 26 may be part of a single device. For example, the computing device 26 (e.g., a circuit, a processor and memory) may be a component of the Raman spectrum analyzer 22.

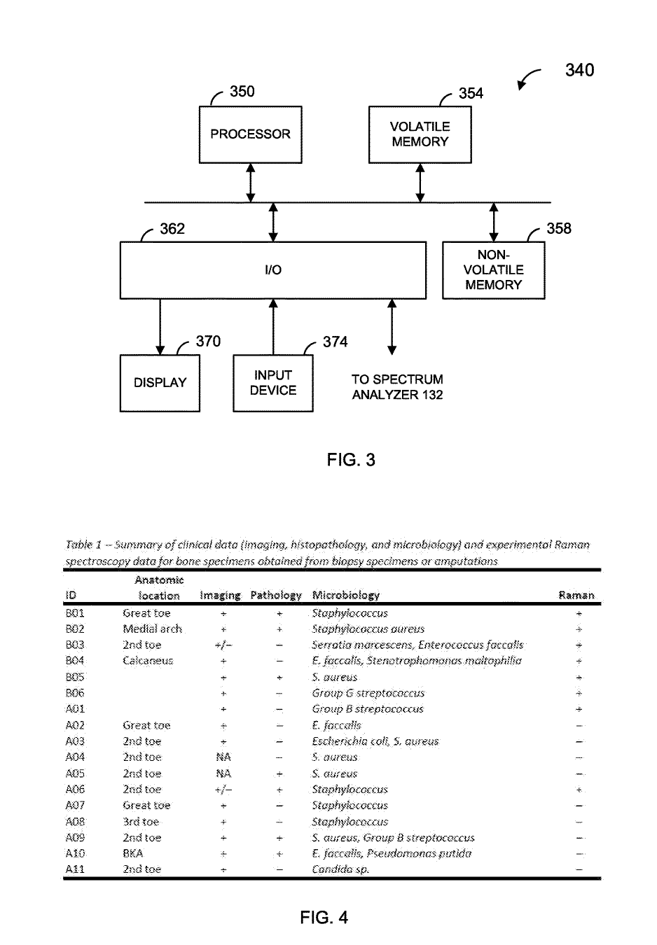

FIG. 3 is a block diagram of an example computing device 26 that may be employed with the example apparatus 10 shown in FIG. 1. It is to be understood that the computer 300 illustrated in FIG. 3 is merely one example of a computing device 26 that may be employed and many other types of computing devices 26 may be used as well. The computer 300 may include at least one processor 302, a volatile memory 304, and a non-volatile memory 306. The volatile memory 304 may include, for example, a random access memory (RAM). The non-volatile memory 306 may include, for example, one or more of a hard disk, a read-only memory (ROM), a CD-ROM, an erasable programmable ROM (EPROM), an electrically erasable programmable ROM (EEPROM), a digital versatile disk (DVD), a flash memory, etc. The computer 300 may also include an I/O device 308. The processor 302, volatile memory 304, non-volatile memory 306, and the I/O device 308 may be interconnected via an address/data bus 310. The computer 300 may also include at least one display 312 and at least one user input device 314. The user input device 314 may include, for example, one or more of a keyboard, a keypad, a mouse, a touch screen, etc. In some embodiments, one or more of the volatile memory 304, non-volatile memory 306, and the I/O device 308 may be coupled to the processor 302 via a bus (not shown) separate from the address/data bus 310, or coupled directly to the processor 302.

The display 312 and user input 314 devices are coupled with the I/O device 308. The computer 300 may be coupled to the spectrum analyzer 22 (FIG. 1) via the I/O device 308. Although the I/O device 308 is illustrated in FIG. 3 as one device, it may comprise several devices. Additionally, in some embodiments, one or more of the display 312 device, the user input device 314, and the spectrum analyzer 22 may be coupled directly to the address/data bus or the processor 302. Additionally, as described previously, in some embodiments the spectrum analyzer 22 and the computer 300 may be incorporated into a single device.

A routine for determining bone tissue infection based on Raman spectral content information may be stored, for example, in whole or in part, in the non-volatile memory 306 and executed on, in whole or in part, by the processor 302. For example, the procedure 100 of FIG. 2 could be implemented in whole or in part via a software program for execution by the processor 302. The program may be embodied in software stored on a tangible medium such as CD-ROM, a floppy disk, a hard drive, a DVD, or a memory associated with the processor 302, and persons of ordinary skill in the art will readily appreciate that the entire program or parts thereof could alternatively be executed by a device other than a processor, and/or embodied in firmware and/or dedicated hardware in a well known manner. With regard to the method 100 of FIG. 2, one of ordinary skill in the art will recognize that the order of execution of the blocks may be changed, and/or the blocks may be changed, eliminated, or combined. Also, although the method 100 of FIG. 2 was described above as being implemented by the computer 300, one or more of the blocks of FIG. 2 may be implemented by other types of devices such as an analog circuit, a digital circuit, a mixed analog and digital circuit, a processor with associated memory, etc.

A clinical study is described in the following appendix.

While the invention is susceptible to various modifications and alternative constructions, certain illustrative embodiments thereof have been shown in the drawings and are described in detail herein. It should be understood, however, that there is no intention to limit the disclosure to the specific forms disclosed, but on the contrary, the intention is to cover all modifications, alternative constructions and equivalents falling within the spirit and scope of the disclosure as defined by the appended claims.

### Appendix

Osteomyelitis of the diabetic foot, herein called diabetic osteomyelitis, is a major cause of lower-extremity amputation, yet an understanding of the pathophysiology and technologies enabling early diagnosis of this serious infection are lacking. Clinical and imaging tests show that whole-tissue properties of bone, including hardness and mineralization, are directly affected by diabetic osteomyelitis. We hypothesized that compositional changes to bone mineral and collagen matrix accompany clinically observable alterations in bone hardness and mineralization. However, no studies to our knowledge have reported on the chemical composition of bone in diabetic osteomyelitis. The objective of the present study was to measure bone composition in diabetic osteomyelitis with the use of Raman spectroscopy.

### RESEARCH DESIGN AND METHODS

### Clinical study

Bone was obtained from 17 patients with a clinical diagnosis of diabetic osteomyelitis requiring surgical intervention to collect a bone biopsy specimen (n=6) or to amputate (n=11). No patients were treated with bone cements. Bone fragments were prepared separately for microbiological and histopathological analyses. All patients had bone cultures performed, and some had additional soft tissue and exudates cultured. For microbiology analysis, bone fragments were stored in an ESwab Collection and Transport system (Becton Dickinson, Sparks, Md.) and analyzed through standard hospital procedure. Fragments for histopathology were prepared by the UMHS Tissue Procurement Core or the AAVA pathology laboratory. Only otherwise-to-be-discarded bone fragments were used for research purposes.

### Bone fragment preparation

Bone fragments for Raman spectroscopic analysis were transported and stored in gauze soaked with PBS enriched with protease inhibitor (0.1% volume for volume) and sodium azide (0.005% weight for volume) to prevent enzymatic or bacterial digestion of bone collagen and stored at −20° C. until examination. Most specimens were examined by Raman spectroscopy within 24 h of the biopsy or amputation surgery and thawed at room temperature immediately before analysis. The average size of the biopsy specimens was <5 mm3, and the average size of the amputation specimens was >1 cm3. Raman spectra were collected with microscopy instrumentation adapted for Raman microspectroscopy as described elsewhere.

Results—Table 1 (FIG. 4) shows the clinical imaging, pathology, microbiology, and Raman spectroscopy data for all study participants. In most cases, multiple clinical imaging modalities (magnetic resonance imaging, X ray, ultrasound, or bone scan) were used for preoperative identification of osteomyelitis. Pathology data on a range of pathophysiological states were reactive, active remodeling, necrotic, or osteomyelitic bone. Additional histopathological findings of acute inflammation or fibrosis were found in a few participants in the amputation group. As expected, bone cultures revealed a mixed population of gram-positive bacteria, with Staphylococcus, Streptococcus, or Enterococcus as the dominant species. Raman spectroscopy of the bone fragments revealed the presence of pathological minerals in addition to normal bone mineral. Two pathological minerals were identified: brushite and uncarbonated apatite. A + for Raman spectroscopic results was reported if brushite or uncarbonated apatite was detected. Raman spectra of control bone specimens were consistent with normal bone composition and did not show evidence of pathological mineralization. Storage in enriched PBS did not affect induced compositional changes in a control study of healthy bone fragments.

Clinical evaluation of study participants also included age, sex, height, weight, disease duration, and history of foot ulcers. Study participants were 41-87 years old. The biopsy cohort comprised two women and four men, and the amputation cohort comprised 11 men. In most cases, the affected foot was assessed by X ray, magnetic resonance imaging, bone scan, or ultrasound imaging within 1 month of the biopsy or amputation. If known, the anatomic location of the surgery or biopsy is included. In several cases, multiple clinical imaging modalities were used to ascertain the presence of osteomyelitis, and any diagnostic radiology report is identified with a + in Table 1 (FIG. 4). In a few cases, multiple clinical imaging tests did not yield consistent or unambiguous preoperative identification of osteomyelitis. For those cases, the results are reported from the positive test. Inconclusive or ambiguous diagnostic radiology reports are identified with a +/2. A + value for pathology results was reported only if the histopathological diagnosis was either acute or chronic osteomyelitis. Positive histopathology reports included evidence of bone remodeling, inflammation, necrosis, the presence of reactive bone, and osteolysis. As expected, Staphylococcus, Streptococcus, and Enterococcus were the primary bacterial species recovered from bone cultures. Raman identification of abnormal minerals, either brushite or uncarbonated apatite, are also denoted with a +. Hypercalcemia and chronic metabolic acidosis were ruled out as a possible cause of pathological mineralization because all participants had normal-to-low serum calcium levels and normal serum bicarbonate levels. A, amputation; B, biopsy; BKA, below-the-knee amputation; NA, not available.

Conclusions—In this study, we applied Raman spectroscopy to measuring compositional changes in bone infected by osteomyelitis of the diabetic foot. Bone fragments were examined from patients who underwent either surgical biopsy/debridement or amputation. An unexpected finding was Raman spectral patterns corresponding to dicalcium phosphate dihydrate, also called brushite, and uncarbonated apatite. Compositional changes in bone currently cannot be identified by standard clinical imaging or histopathology but are easily measured by Raman spectroscopy. This study provides insight into the pathophysiology of diabetic osteomyelitis and identified a possible early-stage marker of clinical disease.

Many mechanisms of bone loss in osteomyelitis have been proposed in the literature. Even though bacterial biofilms are known to form in osteomyelitis, direct bacterial attack on bone is believed to be a negligible mechanism. The present results suggest that pathological mineralization accompanies bacterial infection, providing insight into the pathophysiology of osteomyelitis. The presence of pathological minerals may also serve as a compositional marker of early-stage bone infection. Brushite is only found in vivo under chronically acidic conditions, such as dental calculus, urinary stones, and chondrocalcinosis. To the best of our knowledge, this is the second report of brushite in mature human bone. Brushite was identified by X-ray absorption and infrared spectroscopy in fibrous dysplasia of the jaw. How-ever, this finding has not been reproduced in other studies, and results from only one patient were reported. Poorly carbonated apatite can be found in woven, or immature, bone and is less crystalline than mature bone mineral. By contrast, the uncarbonated apatite found in infected bone was more crystalline than immature bone mineral and suggests deposition of a pathological mineral.

Normal serum calcium values in all the participants argue against the possibility that we were observing brushite and uncarbonated apatite as a precursor in normal bone formation or as a nonbone precipitate resulting from systemic hypercalcemia. The likelihood that pathological minerals were formed by an inflammatory response, immune response, or excessive bone remodeling is not supported by our observations and previous studies. Thus, we hypothesize that a bacteria bio-film is responsible for generating the acidic environment necessary to form brushite. If the localized microenvironment cannot be adequately buffered, then acidic calcium phosphate minerals such as uncarbonated apatite and brushite may precipitate onto the bone surface. This mechanism, although new in its application to diabetic osteomyelitis, is the accepted pathway in microbial degradation of bone postmortem.

Associating Raman spectroscopy data with anatomic location was an issue in the measurements and may have had an impact on the rate of identifying pathological minerals. Biopsy specimens were small (<5 mm3) and taken directly from the wound bed, so there was a greater association between the spectroscopy data and the anatomic location of the active infection. Thus, we were able to identify pathological minerals in 100% of the biopsy specimens. However, the amputated tissue was large relative to the recovered fragments. Although we worked closely with the pathology laboratory to obtain bone specimens near the site of suspected infection, obtaining precise anatomic information was a challenge. This challenge was also apparent when we examined the imaging and histopathology data. The lack of correlation between imaging and histopathology data in the amputation cohort underscores the difficulty in identifying osteomyelitis across a large anatomical unit, such as a digit or limb. We suspect that incomplete sampling was primarily responsible for inconsistent Raman spectroscopic identification of pathological minerals in amputated bone. Future translational studies will address developing enhanced anatomic precision with respect to geographic analysis of diabetic wounds.

It is intriguing to conceptualize an at-patient Raman spectroscopic measurement of pathological mineralization. Intraoperative or transcutaneous Raman spectroscopic identification of pathological minerals during biopsy or amputation surgeries may distinguish bone infections from noninfectious bone lesions. Point-of-care measurements are feasible because Raman spectroscopy is amenable to fiber-optic-based instrumentation. Our laboratory has developed portable fiber-optic instrumentation for transcutaneous bone measurements at bedside or in a surgical suite, and our ongoing human studies demonstrate in vivo feasibility and establish a basis for future translational Raman studies of diabetic foot wounds.

What is claimed is:

1. A method for determining a condition of a bone tissue, the method comprising:
obtaining a specimen associated with bone tissue suspected of being infected;
monitoring the specimen for formation of brushite or uncarbonated apatite;
indicating a presence of infection in response to detection of brushite or uncarbonated apatite within the specimen.

2. A method as defined in claim 1, wherein obtaining a specimen comprises:
swabbing a sample material from an area proximate the bone tissue; and,
placing the sample material within an apatite-impregnated aqueous growth medium.

3. A method as defined in claim 2, wherein monitoring a specimen for formation of brushite or uncarbonated apatite comprises:
irradiating the sample material and apatite-impregnated aqueous growth medium with a light source;
receiving light scattered from the irradiated sample material and apatite-impregnated aqueous growth medium;
determining Raman spectral content information associated with the received scattered light; and
analyzing the Raman spectral content information to determine a presence of brushite or uncarbonated apatite.

4. A method as defined in claim 3, wherein irradiating the sample material and apatite-impregnated aqueous growth medium comprises using a substantially monochromatic light source.

5. A method as defined in claim 4, wherein the apatite-impregnated growth medium includes an antibiotic agent.

6. A method as defined in claim 5, wherein the antibiotic agent facilitates differentiation of gram-positive or gram-negative bacteria.

7. A method as defined in claim 1, wherein the specimen is a portion of bone tissue in vivo.

8. A method as defined in claim 7, wherein monitoring a specimen for formation of brushite or uncarbonated apatite comprises:
irradiating a portion of the specimen using a light source;
receiving light scattered from the irradiated portion of the specimen;
determining Raman spectral content information associated with the received scattered light; and
analyzing Raman spectral content information to determine presence of brushite or uncarbonated apatite.

9. A method as defined in claim 8, wherein irradiating a portion of the specimen comprises using a substantially monochromatic light source.

10. A method as defined in claim 1, wherein the specimen is a portion of bone tissue ex vivo.

11. A method as defined in claim 10, wherein monitoring a specimen for brushite or uncarbonated apatite formation comprises:
irradiating a portion of the specimen using a light source;
receiving light scattered from the irradiated portion of the specimen;
determining Raman spectral content information associated with the received scattered light; and
analyzing Raman spectral content information to determine presence of brushite or uncarbonated apatite.

12. A method as defined in claim 11, wherein irradiating a portion of the specimen comprises using a substantially monochromatic light source.

13. A method as defined in claim 10, wherein obtaining a specimen comprises:
obtaining a sample material swabbed from an area proximate the portion of bone tissue; and
placing the sample material within an apatite-impregnated aqueous growth medium.

14. A method as defined in claim 13, wherein monitoring a specimen for formation of brushite or uncarbonated apatite comprises:
irradiating the specimen using a light source;
receiving light scattered from the irradiated specimen;
determining Raman spectral content information associated with the received scattered light; and
analyzing Raman spectral content information to determine presence of brushite or uncarbonated apatite.

15. A method for determining whether a bone tissue is infected, the method comprising:
obtaining a sample material from an area proximate the bone tissue;
placing the sample material within a carbonated apatite-impregnated aqueous growth medium;
irradiating the sample material and carbonated apatite-impregnated aqueous growth medium with a monochromatic light source;
receiving light scattered from the irradiated sample material and carbonated apatite-impregnated aqueous growth medium;
determining Raman spectral content information associated with the received scattered light;
analyzing the Raman spectral content information to detect brushite or uncarbonated apatite resulting from a conversion of carbonated-apatite in the presence of bacteria; and
indicating whether brushite or uncarbonated apatite is present within the material and apatite-impregnated aqueous growth medium.

16. An apparatus for a method of determining whether a bone tissue is infected wherein a sample material associated with the bone tissue is placed within an apatite-impregnated aqueous growth medium and irradiated to detect the presence of brushite or uncarbonated apatite resulting from a conversion of carbonated-apatite in the presence of bacteria, the apparatus comprising:
a substantially monochromatic light source irradiating the sample material;
a light receiver to receive light scattered from the sample material within the apatite-impregnated growth medium irradiated by the substantially monochromatic light source;
a Raman spectrum analyzer optically coupled to receive scattered light received by the light receiver, the Raman spectrum analyzer configured to generate Raman spectral content information associated with the received scattered light;
a computing device communicatively coupled to the Raman spectrum analyzer, the computing device configured to generate diagnostic information indicative of whether brushite or uncarbonated apatite is present within the sample material and apatite-impregnated aqueous growth medium, and
a display device for indicating the presence of brushite or uncarbonated apatite within the sample material and apatite-impregnated aqueous growth medium.

17. An apparatus as defined in claim 16, wherein the light receiver comprises a microscope.

18. An apparatus as defined in claim 16, wherein the light receiver comprises an optical probe.

19. An apparatus as defined in claim 16, wherein the light receiver further comprises at least one optical fiber coupled to the lens.

20. An apparatus as defined in claim 16, wherein the computing device comprises a processor coupled to a memory.

21. A method as defined in any one of claims 1 to 14, wherein analyzing Raman spectral content information to determine presence of brushite or uncarbonated apatite comprises determining the presence of brushite.

22. A method as defined in any one of claims 1 to 14, wherein analyzing Raman spectral content information to determine presence of brushite or uncarbonated apatite comprises determining the presence of uncarbonated apatite.

23. A method as defined in claim 15, wherein analyzing the Raman spectral content information to detect brushite or uncarbonated apatite comprises analyzing the Raman spectral content information to detect brushite and wherein indicating whether brushite or uncarbonated apatite is present comprises indicating whether brushite is present.

24. A method as defined in claim 15, wherein analyzing the Raman spectral content information to detect brushite or uncarbonated apatite comprises analyzing the Raman spectral content information to detect uncarbonated apatite and wherein indicating whether brushite or uncarbonated apatite is present comprises indicating whether uncarbonated apatite is present.

25. The apparatus as defined in any one of claims 16 to 20, wherein the computing device is configured to generate diagnostic information indicative of whether brushite is present, and wherein the display device is for indicating the presence of brushite within the sample and apatite-impregnated aqueous growth medium.

26. The apparatus as defined in any one of claims 16 to 20, wherein the computing device is configured to generate diagnostic information indicative of whether uncarbonated apatite is present, and wherein the display device is for indicating the presence of uncarbonated apatite within the sample and apatite-impregnated aqueous growth medium.

27. A method for determining a condition of a bone tissue, the method comprising:
obtaining a specimen associated with bone tissue suspected of being infected;
monitoring the specimen for formation of atypical calcium phosphate minerals, wherein the atypical phosphate minerals are not found in environments surrounding healthy bone tissue;
indicating a presence of infection in response to detection of atypical phosphate minerals within the specimen.

28. The method as defined in claim 27, wherein monitoring the specimen for formation of atypical calcium phosphate minerals comprises monitoring the specimen for phosphate minerals found at pH values more acidic than physiological pH.

29. The method as defined in claim 27, wherein monitoring the specimen for formation of atypical calcium phosphate minerals comprises monitoring the specimen for carbonated apatite outside the carbonation range of normal bone tissue.

30. A method as defined in claim 27, wherein obtaining a specimen comprises:
swabbing a sample material from an area proximate the bone tissue; and,
placing the sample material within an apatite-impregnated aqueous growth medium.

31. A method as defined in claim 30, wherein monitoring the specimen for formation of atypical calcium phosphate minerals comprises:
irradiating the sample material and apatite-impregnated aqueous growth medium with a light source;
receiving light scattered from the irradiated sample material and apatite-impregnated aqueous growth medium;
determining Raman spectral content information associated with the received scattered light; and
analyzing the Raman spectral content information to determine a presence of atypical calcium phosphate minerals.

32. A method as defined in claim 31, wherein irradiating the sample material and apatite-impregnated aqueous growth medium comprises using a substantially monochromatic light source.

33. A method as defined in claim 32, wherein the apatite-impregnated growth medium includes an antibiotic agent.

34. A method as defined in claim 33, wherein the antibiotic agent facilitates differentiation of gram-positive or gram-negative bacteria.

35. A method as defined in claim 27, wherein the specimen is a portion of bone tissue in vivo.

36. A method as defined in claim 35, wherein monitoring a specimen for formation of atypical calcium phosphate minerals comprises:
irradiating a portion of the specimen using a light source;
receiving light scattered from the irradiated portion of the specimen;
determining Raman spectral content information associated with the received scattered light; and
analyzing Raman spectral content information to determine presence of atypical calcium phosphate minerals.

37. A method as defined in claim 36, wherein irradiating a portion of the specimen comprises using a substantially monochromatic light source.

38. A method as defined in claim 27, wherein the specimen is a portion of bone tissue ex vivo.

39. A method as defined in claim 38, wherein monitoring a specimen for atypical calcium phosphate mineral formation comprises:
irradiating a portion of the specimen using a light source;
receiving light scattered from the irradiated portion of the specimen;
determining Raman spectral content information associated with the received scattered light; and
analyzing Raman spectral content information to determine presence of atypical calcium phosphate minerals.

40. A method as defined in claim 39, wherein irradiating a portion of the specimen comprises using a substantially monochromatic light source.

41. A method as defined in claim 38, wherein obtaining a specimen comprises:
obtaining a sample material swabbed from an area proximate the portion of bone tissue; and
placing the sample material within an apatite-impregnated aqueous growth medium.

42. A method as defined in claim 41, wherein monitoring a specimen for formation of atypical calcium phosphate minerals comprises:
irradiating the specimen using a light source;
receiving light scattered from the irradiated specimen;
determining Raman spectral content information associated with the received scattered light; and
analyzing Raman spectral content information to determine presence of atypical calcium phosphate minerals.
